(19) United States
(12) Patent Application Publication
(10) Pub. No.: US 2023/0271142 A1
(43) Pub. Date: Aug. 31, 2023

(54) METHODS OF PREPARING SOLID PARTICULATE MATERIALS

(71) Applicant: MICROPORE TECHNOLOGIES LIMITED, Cleveland (GB)

(72) Inventors: Elina Beveniou, Cleveland (GB); Matt Bennett, Cleveland (GB); Alex Kerr, Cleveland (GB); Sam Trotter, Cleveland (GB); David Hayward, Cleveland (GB)

(73) Assignee: MICROPORE TECHNOLOGIES LIMITED, Cleveland (GB)

(21) Appl. No.: 18/018,078

(22) PCT Filed: Jul. 29, 2021
(86) PCT No.: PCT/GB2021/051965

§ 371 Date: Jan. 26, 2023

(30) Foreign Application Priority Data
Jul. 30, 2020 (GB) 2011836.0

(51) Int. Cl. B01D 69/02; A61K 31/40; A61K 31/4184; B01D 67/00; B01D 69/04; B01D 71/02; B01D 9/00; B01F 23/451

(52) U.S. Cl. CPC B01D 69/02; A61K 31/40; A61K 31/4184; B01D 67/0055; B01D 69/04; B01D 71/022; B01D 9/0013; B01D 9/0054; B01D 9/0063; B01F 23/451; B01D 2325/021

(57) ABSTRACT

There is described a method of preparing solid particles of a compound, said method comprising controlling provision of a liquid phase, wherein said liquid phase comprises a solution of the compound, in a first flow direction to a membrane, said membrane defining a plurality of pores; and controlling the supersaturation of the liquid phase after it has passed through the membrane via the plurality of pores, to form solid particles of the compound. The method may comprise a continuous method.

### FIELD OF THE INVENTION

The present invention relates to a novel method of preparing solid particulate materials.

More particularly, the present invention relates to a method of preparing solid particulate compounds for use in a variety of fields, including, but not limited to, pharmaceutically active compounds.

### BACKGROUND TO THE INVENTION

Crystallisation or re-crystallisation is a technique often used for the purification of chemical compounds, or for the control of the form and size of solid material.

By dissolving a compound and any accompanying impurities, often impurities related to the process of preparation of the compound, in an appropriate solvent, either the desired compound or impurities can be removed from the solution, leaving the other behind.

Precipitation or crystallisation requires lower energy than other separation processes. Most crystallisation applications in the chemical field involve precipitation of a compound from a solution by directly or indirectly cooling the solution and/or by evaporating part of the solvent in order to effect crystallisation. For example, many inorganic salts are made industrially from aqueous solutions which are produced by dissolving a natural source of the salt in water. The salt is usually obtained by crystallising it from the aqueous solution by evaporation of the water. However, such evaporation processes are energy intensive. If the separation of a salt from water could be done without vaporising water, substantial energy savings would be possible.

Another method for the crystallisation of chemical compounds is by an antisolvent crystallisation process. In an antisolvent process the compound which is to be crystallised is dissolved in a solvent and precipitation is induced by the addition of second solvent in which the compound is insoluble or poorly soluble, an antisolvent. The solvents are selected such that the compound of interest is partially soluble in one solvent, referred to as “the solvent” and substantially insoluble in the other solvent, referred to as “the anti-solvent”. The term “anti-solvent” is used herein to describe a solvent that the compound(s) of interest shows a substantially lower solubility in.

Antisolvent crystallisation can be an energy saving alternative to evaporative crystallisation processes. A solution containing the compound becomes supersaturated. Generally, the metastable solubility limit is not breached, and large crystals can be formed.

Solid particulate materials produced by antisolvent crystallisation may comprise crystalline or amorphous particles, or a combination thereof.

Generally, crystalline solids have regular ordered arrays of components held together by uniform intermolecular forces in a crystal lattice, a repeating three-dimensional structure. Whereas the components of amorphous solid particles are aggregated with no particular order.

Crystalline solids have distinctive internal structures and distinctive surfaces or faces. The faces intersect at angles that are characteristic of the substance. When exposed to x-rays, each structure produces a distinctive pattern that can be used to identify the material. In addition, crystalline solids tend to have relatively sharp, well-defined melting points.

Generally, the characteristics of amorphous solids are that when cleaved or broken, amorphous particles produce fragments with irregular surfaces; and they have poorly defined patterns when exposed to x-rays because their components are not arranged in a regular array. In addition, amorphous solids tend do not have a definite melting points; amorphous solids melt gradually over a range of temperatures. Furthermore, it has been disclosed that the amorphous forms in a number of drugs exhibit different dissolution characteristics and in some cases different bioavailability patterns compared to the crystalline form (Konno T., Chem. Pharm Bull., 1990; 38:2003-2007).

Two non-limiting examples of amorphous pharmaceutically active compounds in which there has been much interest are atorvastatin, described in International Patent application No. WO 97/039060 (Warner Lambert); and telmisartan, described in US patent application No. 2006/0111417 (Dr Reddy's).

The Biopharmaceutics Classification System (BCS) is a system to differentiate the drugs on the basis of their solubility and permeability. According to the BCS, drug substances are classified into four classes upon their solubility and permeability:
Class I—high permeability, high solubility
Class II—high permeability, low solubility
Class III—low permeability, high solubility
Class IV—low permeability, low solubility

Many pharmaceutically active compounds have low aqueous solubility. Over 60% of APIs (Active Pharmaceutical Ingredients) fall under BCS Class II or Class IV. Such APIs usually suffer from poor bioavailability and incomplete/erratic absorption.

Bioavailability of APIs can be improved by, inter alia, increasing the surface area of a compound. The surface area of a compound can be increased by a variety of techniques including, mechanical milling, high pressure homogenization or spray drying. However, these techniques require high energy inputs and expensive equipment; and often lead to thermal degradation, heterogeneous particle shapes, and the like.

Antisolvent precipitation techniques have been used as an alternative approach for the preparation of APIs with low permeability and/or low solubility.

In reverse antisolvent precipitation, the solution containing the compound is added to the antisolvent. As the antisolvent has a very low tolerance to the solute, the solution becomes supersaturated and exceeds the metastable limit very quickly. With reverse antisolvent precipitation the process is nucleation controlled, leading to many, small solid particles.

More recently, the US Food and Drug Administration (FDA) and other regulatory agencies have set strict standards to ensure the safety and stability of pharmaceuticals and generally higher requirements for medicine production and particularly for the crystallisation process are being set. Consequently, crystallisation is developing from an empirical science to an evidence- and theory-based science.

US Patent application No. 2006/0182808 describes an antisolvent precipitation process wherein a liquid medium comprising a compound to be solidified is forced through a membrane into an antisolvent, or wherein an antisolvent is forced through a membrane into a liquid medium comprising a compound which is to be solidified, yielding a composition comprising solid particles of the compound.

Othman et al, “Preparation of Microcrystals of Piroxicam Monohydrate by Antisolvent Precipitation via Microfabricated Metallic Membranes with Ordered Pore Arrays”, Crystal Growth & Design, 2017, 17, 6692-6702, describes the preparation of microcrystals of piroxicam (PRX) monohydrate with a narrow size distribution from acetone/PRX solutions by antisolvent crystallization via metallic membranes with ordered pore arrays.

International Patent application No. WO 2019/092461 describes a crossflow apparatus for producing an emulsion or dispersion by dispersing a first phase in a second phase.

### SUMMARY OF THE INVENTION

Therefore, there is a need for a method of precipitation of compounds, providing a narrow size distribution of the solidified particles. The method should be capable of being scaled up if desirable and can optionally be continuous process.

Thus, the present invention allows the scale up and/or continuous production of small and non-aggregated solid particles by conventional techniques, e.g. cooling a solution of the compound to effect precipitation, antisolvent precipitation or reverse antisolvent precipitation. It will be understood by the person skilled in the art that antisolvent precipitation and reverse antisolvent precipitation will be especially suitable for those compounds with poor solubility and/or permeability.

Apparatus for use in membrane emulsification usually utilise a two phase dispersion with large droplets is forced though a high shear region to induce turbulence and thereby to break up the drops into smaller ones. However, we have surprisingly found that membrane emulsification apparatus can be utilised to generate laminar mixing of liquid phases.

Furthermore, it has been surprisingly found that a crossflow membrane emulsification apparatus (AXF), utilising a tubular membrane, can suitably be used for the production of solid particles.

According to a first aspect of the invention there is provided a method of preparing solid particles of a compound, said method comprising controlling provision of a liquid phase, wherein said liquid phase comprises a solution of the compound, in a first flow direction to a membrane, said membrane defining a plurality of pores; and controlling the supersaturation of the liquid phase after it has passed through the membrane via the plurality of pores, to form solid particles of the compound.

The supersaturation of the liquid phase may be controlled by any of the methods herein described, for example, cooling the liquid phase, antisolvent precipitation or reverse antisolvent precipitation.

In one aspect of the invention the method comprises cooling the liquid phase after it has passed through the membrane.

In another aspect of the invention the method comprises antisolvent precipitation after the liquid phase has passed through the membrane.

In another aspect of the invention the method comprises reverse antisolvent precipitation after the liquid phase has passed through the membrane.

The method of the invention may comprise the preparation of solid particles of a compound in crystalline form or amorphous form, or a combination thereof. In one aspect of the invention the method comprises the preparation of solid crystalline particles of a compound. In another aspect of the invention the method comprises the preparation of solid amorphous particles of a compound.

According to a further aspect of the invention there is provided a method of preparing solid particles of a compound, said method comprising dispersing a first liquid phase in a second liquid phase;
wherein said first liquid phase comprises a solution of the compound and said second liquid phase comprises an antisolvent; or said first liquid phase comprises an antisolvent and said second liquid phase comprises a solution of the compound;
said method comprising controlling provision of the first liquid phase in a first flow direction to a membrane, said membrane defining a plurality of pores; and controlling provision of the second liquid phase to the membrane in a crossflow (AXF) to the first flow direction, via the plurality of pores, to form solid particles of the compound.

In one aspect of the invention the first liquid phase comprises a solution of the compound and the second liquid phase comprises an antisolvent.

In another aspect of the invention the first liquid phase comprises an antisolvent and the second liquid phase comprises a solution of the compound.

According to a further aspect of the invention there is provided a method of preparing solid particles of a compound, said method comprising dispersing a first liquid phase in a second liquid phase;
wherein said first liquid phase comprises a solution of the compound and said second liquid phase comprises an antisolvent phase; or said first liquid phase comprises an antisolvent and said second liquid phase comprises a solution of the compound;
wherein said method uses a crossflow emulsification apparatus; said crossflow emulsification apparatus comprising:
an outer tubular sleeve provided with a first inlet at a first end; a particle outlet; and a second inlet, distal from and inclined relative to the first inlet;
a tubular membrane provided with a plurality of pores and adapted to be positioned inside the tubular sleeve; and
optionally an insert adapted to be located inside the tubular membrane, said insert comprising an inlet end and an outlet end, each of the inlet end and an outlet end being provided with chamfered region; the chamfered region is provided with a plurality of orifices and a furcation plate; and
controlling provision of the first liquid phase to the tubular membrane; and controlling provision of a second liquid phase to the tubular membrane via the plurality of pores to form solid particles.

In one aspect of the invention the first liquid phase comprises a solution of the compound and the second liquid phase comprises an antisolvent.

In another aspect of the invention the first liquid phase comprises an antisolvent and the second liquid phase comprises a solution of the compound.

It will be understood that is provided the method of the invention may comprise preparing solid particles of more than one compound, e.g. as co-crystals, comprising two or more components, and which may form a unique crystalline structure with unique properties.

The solution will include one or more dissolved materials. A variety of dissolved materials may be subjected to the method of the present invention. Typically, the dissolved material may be one or more organic compounds, which may include, for example, pharmaceutically active compounds, bioactive agents, nutraceuticals, polymers and the like.

This method of the invention may comprise the preparation of solid particles of a compound in crystalline form or amorphous form, or a combination thereof. In one aspect of the invention the method comprises the preparation of solid crystalline particles of a compound. In another aspect of the invention the method comprises the preparation of solid amorphous particles of a compound.

In a particular aspect of the invention dissolved material comprises a material of low solubility. The term “low solubility” should be construed as meaning materials of poor bioavailability due to low water solubility. Up to 90% of the active pharmaceutical substances under development are poorly water soluble, usually resulting in low bioavailability. The term “anti-solvent” is used herein to describe a solvent or a mixture of solvents wherein a compound of interest shows a substantially lower solubility when compared with the solvent or in which the compound of interest is completely insoluble. The term “solvent” is used herein to describe a solvent or a mixture of solvents wherein the compound of interest is at least slightly soluble, as defined by US Pharmacopeia. The person skilled in the art will be able to select a solvent that may be used as a solvent for a particular compound and as an anti-solvent for another compound.

In one aspect of the invention the term “soluble” shall mean from 10 to 30 parts solvent is needed to dissolve 1 part solute. The term “low solubility” shall mean from 100 to 10, 000 parts solvent is needed to dissolve 1 part solute. The term “slightly soluble” shall mean from 100 to 1,000 parts solvent is needed to dissolve 1 part solute. The term “insoluble” shall mean more than 10,000 parts solvent is needed to dissolve 1 part solute. These terms are generally defined by the US Pharmacopeia.

More than 90% of active pharmaceutical ingredients (APIs) being developed fall under BCS Class II or Class IV; yet such APIs usually suffer from poor bioavailability and incomplete/erratic absorption.

In another aspect of the invention the compound of interest is a compound that lies within Class II or IV of the Biopharmaceutics Classification System (BCS). In one aspect of the invention the compound of interest is a compound that lies within Class II of the BCS. In another aspect of the invention the compound of interest is a compound that lies within Class IV of the BCS.

The ratios and amounts of those compounds may be adjusted according to the compound, solvents, antisolvents and physicochemical properties, such as solubility, melting point, etc.

Typically the volumetric ratio of solution to antisolvent may be from about 1:0.5 to about 1:50, e.g. from about 1:1 to about 1:40 or from about 1:2 to about 1:4. The solution solvents and antisolvents used in the present invention may vary, but are typically those that are acceptable in food, pharmaceutical and cosmetic products and which can be used in the production of solid particles. These include, but shall not be limited to, for example, alcohols, aliphatic and alicyclic alkanes, ethers, esters, hydrocarbons, ketones, water, and the like. Furthermore, the solution solvents and antisolvents include, but shall not be limited to, for example, ethanol, water, hexane, glycerol, t-butanol, isopropanol, ethyl acetate, and the like.

Desirably the solvent phase and the antisolvent phase may be substantially miscible or partly miscible with one another.

Mixtures of two or more solvents and/or two or more antisolvents may be used to more readily control the production of the solid particles.

Crystallisation can be affected by the addition of surfactants, which may play a role in nucleation and growth kinetics and may modify size distribution of crystalline and amorphous particles. In addition, the addition of surfactants may modify the crystal polymorph and particle morphology. The solution solvent and/or the antisolvent may additionally comprise one or more surfactants or co-surfactants.

The surfactants may be selected from one or more of non-ionic surfactants, anionic surfactants, cationic surfactants and zwitterionic surfactants; and combinations thereof.

Non-ionic surfactants used in the present invention may be selected from, but shall not be limited to, polyvinyl alcohol (PVA); hydroxy propyl methyl cellulose (HPMC); poly(ethylene glycol)-block-poly(propylene glycol)-block-poly(ethylene glycol); Pluronic P123 (PEO-PPO-PEO); ethoxylates, including fatty alcohol ethoxylates, such as, octaethylene glycol monodecyl ether, pentaethylene glycol monodecyl ether and hexoxy ethylene glycol mono-n-dodecyl ether; alkylphenol ethoxylates, such as, Triton X-100; fatty acid esters, such as, glycerol monostearate and glycerol monolaurate; fatty acid esters of sorbitol, such as, sorbitan monolaurate, sorbitan monostearate and sorbitan tristearate; fatty acid amides, such as, cocamide monoethanolamine and cocamide diethanolamine; and Tween, e.g. Tween 20, a non-ionic detergent widely used in biochemical applications, Tween 40, Tween 60 and Tween 80; and ethoxylates, including fatty alcohol ethoxylates, such as, octaethylene glycol.

Anionic surfactants may be selected from, but shall not be limited to, sodium dodecyl sulfate (SDS), sodium dodecyl benzene sulfonate (SDBS), ammonium lauryl sulfate, and sodium bis (2-ethyl hexyl) sulfosuccinate.

Cationic surfactants may be selected from, but shall not be limited to, ammonium salts, such as, cetyl trimethyl ammonium bromide (CTAB), cetylpyridinium chloride (CPC), benzalkonium chloride (BAC), benzethonium chloride (BZT), dimethyl dioctadecyl ammonium chloride, dioctadecyl dimethyl ammonium bromide (DODAB) and dodecyl dimethyl ammonium bromide (DDAB).

Zwitterionic surfactants may be selected from, but shall not be limited to phospholipids, such as, phosphatidyl serine, phosphatidyl choline (PC) and phosphatidyl ethanolamine (PE).

The amount of surfactants that is required for achieving good particle size and shape may vary, but may be from about 0.005% to 2.0% w/w of the total solution.

Surfactants and co-surfactants include, but shall not be limited to, for example, Tween, a non-ionic detergent widely used in biochemical applications. It is also known as PEG(20) Sorbitan monolaurate. Other emulsifiers include poloxomer, a hydrophilic non-ionic surfactant which is a non-ionic triblock copolymer, Tween 80 and lecithins.

In the method of the present invention the crossflow membrane emulsification uses the flow of a second phase, to detach droplets from the membrane to sweep and evenly mix flows of a first phase coming through the membrane pores. This contrasts with the use of turbulent flow, e.g. by stirring, for solid particle production.

The position of the particle outlet may vary depending upon the direction of flow of the first liquid phase, i.e. from inside the membrane to outside or from outside the membrane to inside. If the flow of the first liquid phase is from outside the membrane to inside then the particle outlet will generally be at a second end of the tubular sleeve. If the flow of the first liquid phase is from inside the membrane to outside then the particle outlet may be a side branch or at the end.

In one aspect of the invention the crossflow apparatus includes an insert as herein described and the first inlet is a first phase first inlet and the second inlet is a second phase inlet; such that the first phase travels from outside the tubular membrane to inside.

In another aspect of the invention the crossflow apparatus does not include an insert and the first inlet is a first phase inlet and the second inlet is a second phase inlet; such that the first phase travels from inside the tubular membrane to outside.

In one aspect of the invention the first phase is the solution phase and the second phase is an antisolvent phase. The solution solvent phase may optionally include one or more active agents as herein defined.

In another aspect of the invention the first phase is the antisolvent phase and the second phase is a solution phase. The solution solvent phase may optionally include one or more active agents as herein defined.

When an insert is present and the tubular membrane is positioned inside the outer sleeve, the spacing between the insert and the tubular membrane may be varied, depending upon the laminar conditions desired, etc. Generally, the insert will be located centrally within the tubular membrane, such that the spacing between the insert and the membrane will comprise an annulus, of equal or substantially equal dimensions at any point around the insert. Thus, for example, the spacing may be from about 0.05 to about 10 mm (distance between the outer wall of the insert and the inner wall of the membrane), from about 0.1 to about 10 mm, from about 0.25 to about 10 mm, or from about 0.5 to about 8 mm, or from about 0.5 to about 6 mm, or from about 0.5 to about 5 mm, or from about 0.5 to about 4 mm, or from about 0.5 to about 3 mm, or from about 0.5 to about 2 mm, or from about 0.5 to about 1 mm.

When the tubular membrane is positioned inside the outer sleeve, the spacing between the tubular membrane and the outer sleeve may be varied, depending upon the size of droplets desired, etc. Generally, the tubular membrane will be located centrally within the outer sleeve, such that the spacing between the membrane and the sleeve will comprise an annulus, of equal or substantially equal dimensions at any point around the tubular membrane. Thus, for example, the spacing may be from about 0.5 to about 10 mm (distance between the outer wall of the membrane and the inner wall of the sleeve), or from about 0.5 to about 8 mm, or from about 0.5 to about 6 mm, or from about 0.5 to about 5 mm, or from about 0.5 to about 4 mm, or from about 0.5 to about 3 mm, or from about 0.5 to about 2 mm, or from about 0.5 to about 1 mm.

In an alternative embodiment of the invention the insert is tapered, such that the spacing between the insert and the tubular membrane may be divergent along the length of the membrane. The spacing and the amount of divergence varied, depending upon the gradient of the tapered insert, the laminar conditions/flow velocities desired, size distribution, etc. It will be understood by the person skilled in the art that depending upon the direction of taper, the spacing between the insert and the tubular membrane may be divergent or convergent along the length of the membrane. The use of a tapered insert may be advantageous in that a suitable taper may allow the laminar flow to be held constant for a particular formulation and set of flow conditions. Thus, the tapered insert may be used to control variation in drop size resulting from changes in fluid properties, such as viscosity, as the material concentration in the solvent increases through its path along the length of the membrane.

In an alternative embodiment of the invention the crossflow apparatus may comprise more than one tubular membrane located inside the outer tubular sleeve, i.e. a plurality of tubular membranes. When a plurality of tubular membranes is provided, each membrane may optionally have an insert, as herein described, located inside it. A plurality of membranes may be grouped as a cluster of membranes positioned alongside each other. Desirably the membranes are not in direct contact with each other. It will be understood that the number of membranes may vary depending upon, inter alia, the nature of the droplets to be produced. Thus, by way of example only, when a plurality of tubular membranes is present, the number of membranes may be from 2 to 100.

The inclined second inlet provided in the outer tubular sleeve will generally comprise a branch of the tubular sleeve and may be perpendicular to the longitudinal axis of the tubular sleeve. The position of the branch or second inlet may be varied and may depend upon the plane of the membrane. In one embodiment the position of the branch or second inlet will be substantially equidistant from the inlet and the outlet, although it will be understood by the person skilled in the art that the location of this second inlet may be varied. It is also within the scope of the present invention for more than one branch inlet to be provided. For example the use of a dual branch may suitably allow for bleeding the second phase during priming, or flushing for cleaning, or drainage/venting for sterilisation.

The inlet and outlet ends of the outer sleeve will generally be provided with a seal assembly. Although the seal assemblies at the inlet and outlet ends of the outer sleeve may be the same or different, preferably each of the seal assemblies is the same. Normal O-ring seals involve the O-ring being compressed between the two faces on which the seal is required—in a variety of geometries. Commercially available O-ring seals are provided with different groove options with standard dimensions. Each seal assembly will comprise a tubular ferrule provided with a flange at each end. A first flange, located at the end adjacent to the outer sleeve (when coupled) may be provided with a circumferential internal recess which acts as a seat for an O-ring seal. When the O-ring seal is in place, the O-ring seal is adapted to be located around the end of the insert (when present) and within a recess in the outer sleeve to seal against leakage of fluid from within any of the elements of the crossflow apparatus. However, the O-ring seal used in the present invention is designed to allow a loose fit as the membrane slides through the O-rings. This arrangement is advantageous in that it avoids two potential problems while installing the membrane tube:
(1) the potential for crushing the thin membrane tube during installation; and
(2) the potential for the thin membrane tube to cut off the curved surface of the O-ring.

With the O-ring seal used in the present invention, when the end ferrules are clamped onto the outer sleeve they squeeze the sides of the O-rings causing them to deform and press onto the outer surface of the tubular membrane and the inner surface of the sleeve, to form a seal. This requires careful dimensioning and tolerances.

However, it will be understood by the person skilled in the art that other means of making seal may suitably be used, for example, use of a screwed fitting tightened to a particular torque which would avoid the need for close tolerances; or clamping parts to a particular force followed by welding (which may be particularly suitable when using a plastic crossflow apparatus).

The internal diameter of the tubular membrane may be varied. In particular, the internal diameter of the tubular membrane may vary depending upon whether or not an insert is present. Generally, the internal diameter of the tubular membrane will be fairly small. In the absence of an insert the internal diameter of the tubular membrane may be from about 1 mm to about 10 mm, or from about 2 mm to about 8 mm, or from about 4 mm to about 6 mm. When the tubular membrane is intended for use with an insert, the internal diameter of the tubular membrane may be from about 5 mm to about 50 mm, or from about 10 mm to about 50 mm, or from about 20 mm to about 40 mm, or from about 25 mm to about 35 mm. Higher internal diameter of the tubular membrane may only be capable of being subjected to lower injection pressure. The upper limit of the internal diameter of the tubular membrane may depend upon, inter alia, the thickness of the membrane tube, since the cylinder needs to be able to cope with the external injection pressure, and whether it's possible to drill consistent holes through that thickness. The chamber inside the cylindrical membrane usually contains the second phase liquid.

In contrast to membrane emulsification using oscillating membranes, in the present invention the membrane, the sleeve and the insert are generally stationary.

As described herein in prior art membranes, such as those described in WO2012/094595 comprise pores in the membrane that are conical or concave in shape. One example is that the pores in the membrane can be laser drilled. Laser drilled membrane pores or through holes will be substantially more uniform in pore diameter, pore shape and pore depth. The profile of the pores may be important, for example, a sharp, well defined edge around the exit of the pore is preferable. It may be desirable to avoid a convoluted path (such as results from sintered membranes) in order to minimise blockage, reduce feed pressures (cf. mechanical strength), and keep an even flowrate from each pore. However, as discussed herein, it is within the scope of the present invention to use pores in which the internal bore is non-circular (for example rectangular slots) or convoluted (for example tapered or stepped diameter to minimise pressure drop).

In the membrane the pores may be uniformly spaced or may have a variable pitch. Alternatively, the membrane pores may have a uniform pitch within a row or circumference, but a different pitch in another direction.

The pores in the membrane may vary. By way of example only, the pores in the membrane may have a pore diameter of from about 1 μm to about 100 μm, or about 10 μm to about 100 μm, or about 20 μm to about 100 μm, or about 30 μm to about 100 μm, or about 40 μm to about 100 μm, or about 50 μm to about 100 μm, or about 60 μm to about 100 μm, or about 70 μm to about 100 μm, or about 80 μm to about 100 μm, or about 90 μm to about 100 μm. In a further embodiment of the invention the pores in the membrane may have a pore diameter of from about 1 μm to about 40 μm, e.g. about 3 μm, or from about 5 μm to about 20 μm, or from about 5 μm to about 15 μm.

In the membrane the shape of the pores may be substantially tubular. However, it is within the scope of the present invention to provide a membrane with uniformly tapered pores. Such uniformly tapered pores may be advantageous in that their use may reduce the pressure drop across the membrane and potentially increase throughput/flux. It is also within the scope of the present invention to provide a membrane in which the diameter is essentially constant, but the internal bore is non-circular (for example rectangular slots) or convoluted (for example tapered or stepped diameter to minimise pressure drop), providing pores with a high aspect ratio.

The interpore distance or pitch may vary depending upon, inter alia, the pore size; and may be from about 1 μm to about 5,000 μm, or from about 1 μm to about 1,000 μm, or from about 2 μm to about 800 μm, or from about 5 μm to about 600 μm, or from about 10 μm to about 500 μm, or from about 20 μm to about 400 μm, or from about 30 μm to about 300 μm, or from about 40 μm to about 200 μm, or from about 50 μm to about 100 μm, e.g. about 75 μm.

The surface porosity of the membrane may depend upon the pore size and may be from about 0.001% to about 20% of the surface area of the membrane; or from about 0.01% to about 20%, or from about 0.1% to about 20%, or from about 1% to about 20%, or from about 2% to about 20%, or from about 3% to about 20%, or from about 4% to about 20%, or from about 5% to about 20, or from about 5% to about 10%.

The arrangement of the pores may vary depending upon, inter alia, pore size, throughput, etc. Generally, the pores may be in a patterned arrangement, which may be a square, triangular, linear, circular, rectangular or other arrangement. In one embodiment the pores are in a square arrangement.

It will be understood that the apparatus of the invention; and in particular the membrane, may comprise known materials, such as glass; ceramic; metal, e.g. stainless steel or nickel; polymer/plastic, such as a fluoropolymer; or silicon. The use of metals, such as stainless steel or nickel, or polymer/plastic, such as a fluoropolymer is advantageous in that, inter alia, the apparatus and/or membranes may be subjected to sterilisation, using conventional sterilisation techniques known in the art, including gamma irradiation where appropriate. The use of polymer/plastic material, such as a fluoropolymer, is advantageous in that, inter alia, the apparatus and/or membrane may be manufactured using injection moulding techniques known in the art.

As described herein an insert may be included in the membrane to facilitate even flow distribution. However, it is within the scope of the crossflow apparatus of the present invention for the insert to be absent. When an insert is present, the furcation plate may be adapted to split the flow of second phase or the first phase into a number of branches. Whether the furcation plate splits the second phase or the first phase will depend upon the direction of flow of the second phase, i.e. whether the second phase flows through the first inlet or the second inlet. Although the number of furcation plates may be varied, the number selected should be suitable lead to even flow distribution and (at the particle outlet end) not have excessive shear. Preferably, when the insert is present the furcation plate is a bi-furcation plate or a tri-furcation plate to provide a uniform second phase flow within the annular region between the insert and the membrane. Most preferably the furcation plate is a tri-furcation plate.

The number of orifices provided in the insert may vary depending upon the injection rate, etc. Generally the number of orifices may be from 2 to 6. Preferably the number of orifice is three.

The chamfered region on the insert is advantageous in that it enables the insert to be centred when it is located in position inside the membrane. The external circumference of the ends of the insert has a minimal tolerance with the internal diameter of the tubular membrane. This enables the insert to be accurately centred, thereby providing a consistent annulus leading to a consistent laminar flow. Generally, the chamfered region will comprise a shallow chamfer, which is advantageous in that it evens the flow distribution and allows the use of orifices in the insert with larger cross-sectional area than could be achieved if the flow simply entered through orifices parallel to the axis of the insert. This keeps the fluid velocity down and therefore minimises unwanted pressure losses, and shear on the outlet. The distance between the start of the orifices and the start of the porous region on the tubular membrane allows an even velocity distribution to be established. The radial dimension of the insert is selected to provide an annular depth to provide a certain laminar flow for the flowrates chosen. The axial dimension is designed to generally give a combined orifice area which is greater than both the annular area and the inlet/exit tube area.

The use of membrane emulsification techniques in the preparation of solid particles as herein described may comprise the use of turbulent flow or the use of laminar flow, e.g. by stirring or liquid flow. In a particular aspect of the invention the membrane emulsification technique comprises the use of laminar flow, i.e. whilst generally avoiding or minimising any turbulent flow.

The use of membrane emulsification techniques in the preparation of solid particles as herein described may include the use of one or more pump systems. It will be understood that any conventionally known pumping system for use with membrane emulsification may suitably be used. However, in a particular aspect of the invention the pump system may comprise a gear pump or a peristatic pump; and combinations thereof.

The method of the invention can be used to precisely control the distribution of chemical conditions and mechanical forces so that they are substantially constant on a length scale. Hence, resultant solid particles are more uniform in size, hence with narrow size distribution.

The method of the invention may comprise a batch process or a continuous process. Desirably, the method of the invention may comprise a continuous process.

The membrane emulsification apparatus may comprise a laboratory dispersion cell (LDC), which uses a precision engineered circular membrane, with a stirrer being used to generate the shear required for droplet formation; or a crossflow apparatus (AXF). When the AXF is used in a continuous flow method, it is generally referred to as Continuous Crossflow (CXF).

The solid particle size distribution may be measured by a variety of techniques. An exemplary technique is to measure the solid particle size distribution by laser diffraction, e.g. using a Malvern Mastersizer 2000 (Worcestershire, UK). The relative volume, Vi, of the particles in different size classes i, whose mean diameter di range from 0.01 to 3500 μm, may be used to calculate the volume-weighted mean diameter, d[4,3]:

$$d \vert 4 , 3 \vert = \frac{\sum {V}_{i} {d}_{i}}{\sum {V}_{i}}$$

The size uniformity of the solid particle was estimated using span of a particle size distribution:

$$span = \frac{[ d ( v , 0.9 ) - d ( v , 0.1 ) ]}{d ( v , 0.5 )}$$

where d (v, 0.1), d (v, 0.5), and d (v, 0.9) are the particle diameters at 10 vol %, 50 vol %, and 90 vol % of the cumulative distribution.

In one aspect of the invention the crossflow apparatus includes an insert as herein described and the first inlet is a second phase first inlet and the second inlet is a first phase inlet; such that the first phase travels from outside the tubular membrane to inside.

In another aspect of the invention the crossflow apparatus does not include an insert and the first inlet is a first phase first inlet and the second inlet is a second phase inlet; such that the first phase travels from inside the tubular membrane to outside.

Solid particles prepared by the method of the invention are useful as components in pharmaceutical compositions. These compositions will typically include a pharmaceutically acceptable carrier in addition to the pharmaceutically active solid particles.

Therefore, according to a further aspect of the present invention there is provided a compound in solid particle form prepared by the method herein described. The compound in solid particle form according to this aspect of the invention may be in crystalline form or amorphous form, or a combination thereof. In one aspect of the invention the compound in solid particle form comprises solid crystalline particles. In another aspect of the invention the compound in solid particle form comprises solid amorphous particles.

According to this aspect of the invention the compound in solid particle form, e.g. crystalline or amorphous, may comprise an active agent.

According to this aspect of the invention there is provided a composition comprising an active agent in solid particle form as herein described and a pharmaceutically acceptable excipient, carrier or diluent. The active agent in solid particle form may be in crystalline form or amorphous form, or a combination thereof. In one aspect of the invention the active agent is in crystalline form. In another aspect of the invention the active agent is in amorphous form.

By way of example only, active agents which comprise the solid particles of the present invention include, but shall not be limited to, biologically active agents, such as pharmaceutically active agents, vaccines and pesticides. Biologically active compounds may also include, for example, a plant nutritive substance or a plant growth regulant. Alternatively, the active agent may be non-biologically active, such as, a plant nutritive substance, a food flavouring, a fragrance, and the like.

Pharmaceutically active agents refer to naturally occurring, synthetic, or semi-synthetic materials (e.g., compounds, fermentates, extracts, cellular structures) capable of eliciting, directly or indirectly, one or more physical, chemical, and/or biological effects, in vitro and/or in vivo. Such active agents may be capable of preventing, alleviating, treating, and/or curing abnormal and/or pathological conditions of a living body, such as by destroying a parasitic organism, or by limiting the effect of a disease or abnormality by materially altering the physiology of the host or parasite. Such active agents may be capable of maintaining, increasing, decreasing, limiting, or destroying a physiologic body function. Active agents may be capable of diagnosing a physiological condition or state by an in vitro and/or in vivo test. The active agent may be capable of controlling or protecting an environment or living body by attracting, disabling, inhibiting, killing, modifying, repelling and/or retarding an animal or microorganism. Active agents may be capable of otherwise treating (such as deodorising, protecting, adorning, grooming) a body. Depending upon the effect and/or its application, the active agent may further be referred to as a bioactive agent, a pharmaceutical agent (such as a prophylactic agent, or a therapeutic agent), a diagnostic agent, a nutritional supplement, and/or a cosmetic agent, and includes, without limitation, prodrugs, affinity molecules, synthetic organic molecules, proteinaceous compounds, peptides, vitamins, steroids, steroid analogues, nucleic acids, carbohydrates, precursors thereof and derivatives thereof. Active agents may be ionic, non-ionic, neutral, positively charged, negatively charged, or zwitterionic, and may be used singly or in combination of two or more thereof. Active agents may be water insoluble or water soluble.

A wide variety of pharmaceutically active agents may be utilised in the present invention. Thus, the pharmaceutically active agent may comprise one or more of a polynucleotide, a peptide, a protein, a small organic active agent, a small inorganic active agent and mixtures thereof.

In a particular aspect of the present invention the solid particles produced comprise a pharmaceutically active compound. It will be understood by the person skilled in the art that any suitably poorly soluble pharmaceutically active compound may be used in the method of the invention. Such pharmaceutically active compounds may include, but shall not be limited to, antifungal agents, such as, itraconazole fluoconazole, terconazole, ketoconazole and saperconazole; anti-infective agents, such as griseofulvin and griseoverdin; antibiotics, such as, amoxicillin, azithromycin, cephalexin, cefixime, cefoperazone, ceftriaxone, ciprofloxacin, clarithromycin, clavulanic acid, clindamycin, doxycycline, erythromycin, gentamycin, levofloxacin, meropenem, metronidazole, neomycin, norfloxacin, ofloxacin, ornidazole, oxytctracycline, piperacillin, rifampicin, streptomycin, sulbactam, sulfadiazine, tazobactam, tetracycline and tinidazole; anti malaria drugs, such as, atovaquone and artesunate; protein kinase inhibitors, such as, afatinib, axitinib, bosutinib, cetuximab, crizotinib, dasatinib, erlotinib, fostamatinib, gefitinib, ibrutinib, imatinib, zemurasenib, lapatinib, lenvatinib, mubritinib and nilotinib; immune system modulators, such as, cyclosporine; cardiovascular drugs, such as, digoxin and spironolactone; sterols or steroids, such as, betamethasone; ACE inhibitors, such as, captopril, enalapril, ramipril, quinapril, perindopril, lisinopril, and fosinopril; adenohypophyseal hormones; adrenergic antagonists, such as, phentolamine, phenoxybenzamine, tamsulosin, propranolol, atenolol, metoprolol, timolol and acebutolol; adrenocortical steroids; inhibitors of the biosynthesis of adrenocortical steroids; alpha-adrenergic agonists, such as methoxamine, phenylephrine, methyldopa, norepinephrine; alpha-adrenergic antagonists, such as, phentolamine and phenoxybenzamine; analgesics, such as, aspirin and paracetamol; antipyretics and anti-inflammatory agents, such as, diclofenac, ibuprofen, naproxen and ketoprofen; androgens, local anaesthetics, such as, lidocaine; antiaddictive agents; antiandrogens; antiarrhythmic agents, such as, verapamil and diltiazem; antiasthmatic agents, such as, beclomethasone, budesonide, fluticasone, reproterol, salbutamol and salmeterol; anticholinergic agents, such as, ipratropium and oxybutynin; anticholinesterase agents, such as, donepezil; anticoagulants, such as, dabigatran, rivaroxaban, apixaban, edoxaban and betrixaban; antidiabetic agents, such as, metformin; antidiarrheal agents; antidiuretics; antiemetic and prokinetic agents; antiepileptic agents, such as carbamazepine, gabapentin oxcarbazepine; antiestrogens; antifungal agents; antihypertensive agents, such as, losartan, olmesartan, telmisartan and valsartan; antimicrobial agents; antimigraine agents, such as, zolmitriptan; antimuscarinic agents; antineoplastic agents; antiparasitic agents; antiparkinsons agents, such as, carbidopa and levodopa; antiplatelet agents; antiprogestins; antithyroid agents; antitussives; antiviral agents; antidepressants; azaspirodecanediones; barbiturates; benzodiazepines; benzothiadiazides; beta-adrenergic agonists; beta-adrenergic antagonists; selective adrenergic antagonists; selective agonists; bile salts; butyrophenones; calcium channel blockers; catecholamines and sympathomimetic drugs; cholinergic agonists; cholinesterase reactivators; cognitive enhancers, such as, piracetam; dermatological agents; diphenylbutylpiperidines; diuretics; ergot alkaloids; oestrogens; ganglionic blocking agents; ganglionic stimulating agents; glucocorticoid steroids, such as, dexamethasone and prednisolone; agents for control of gastric acidity and treatment of peptic ulcers; haematopoietic agents; histamines; antihistamine; HMG-CoA reductase inhibitors, e.g. statins, such as, atorvastatin, fluvastatin, lovastatin, pitavastatin, pravastatin, rosuvastatin and simvastatin; -hydroxytryptamine antagonists; hypnotics and sedatives; immunosuppressive agents; laxatives; methylxanthines; monoamine oxidase inhibitors; neuromuscular blocking agents; nutrients or dietary supplements, such as, vitamin B1, vitamin B6 and retinol; organic nitrates; opioid analgesics and antagonists; pancreatic enzymes; phenothiazines; progestins; prostaglandins; agents for the treatment of psychiatric disorders; retinoids; sodium channel blockers; thrombolytic agents; thyroid agents; tricyclic antidepressants; tyrosine kinase inhibitors, such as, axitinib, crizotinib, dasatinib, erlotinib, gefitinib, imatinib, lapatinib, nilotinib, pazopanib, regorafenib, ruxolitinib, sorafenib, sunitinib, vandetanib and vemurafenib; drugs from the group comprising danazol, acyclovir, dapsone, indinavir, lopinavir, nifedipine, nitrofurantoin, phentytoin, ritonavir, saquinavir, sulfamethoxazole, valproic acid, trimethoprin, acetazolamide, azathioprine, iopanoic acid, nalidixic acid, nevirapine, praziquantel, rifampicin, albendazole, amitriptyline, artemether, lumefantrine, chloropromazine, clofazimine, efavirenz, iopinavir, folic acid, glibenclamide, haloperidol, ivermectin, mebendazole, niclosamide, pyrantel, pyrimethamine, sulfadiazine, sulfasalazine, triclabendazole, and cinnarizine; and combinations thereof. Such pharmaceutically active compounds may be in free form or salt form.

In one aspect of the invention the compound in solid particle form is an HMG-CoA reductase inhibitor, e.g. a statin, such as, atorvastatin. According to this aspect of the invention the compound in solid particle form is an HMG-CoA reductase inhibitor in amorphous form, e.g. a statin, such as, atorvastatin.

In another aspect of the invention the compound in solid particle form is an antihypertensive agent, such as, telmisartan. According to this aspect of the invention the compound in solid particle form is an antihypertensive agent in amorphous form, such as, telmisartan.

Particles obtained by the method of the present invention may be formulated into a pharmaceutical composition. Examples of pharmaceutical forms for administration of solid particles prepared using the methods herein described may include solid dosage forms, such as, tablets, capsules, granules, pellets or powders. The compositions obtained may have an enhanced performance including, but not exclusively, supersaturation, improved dissolution rate, improved bioavailability, improved or controlled release, and the like.

In one aspect of the present invention the solid particles are not piroxicam monohydrate microcrystals.

The present invention will now be described by way of example only with reference to the accompanying figures in which:

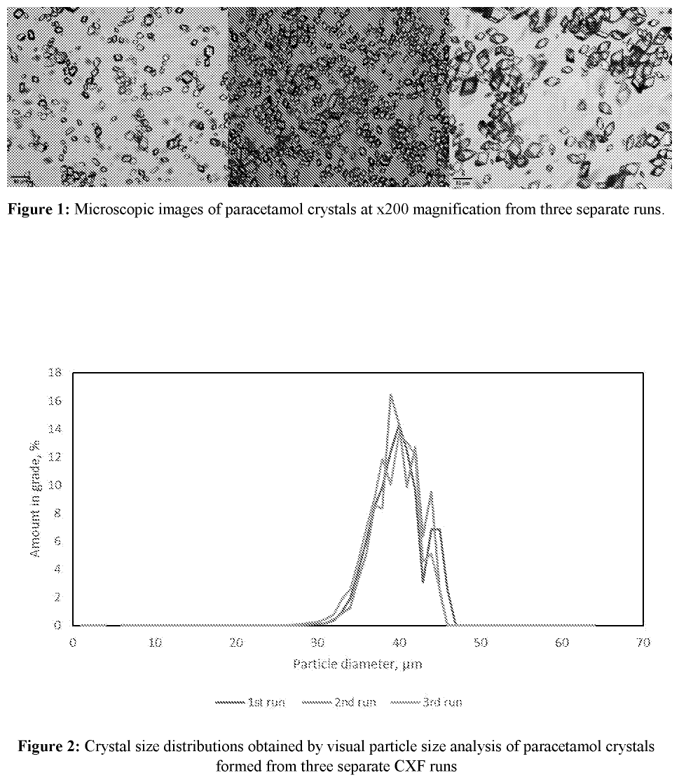

FIG. 1 illustrates microscopic images of paracetamol crystals at ×200 magnification from three separate runs;

FIG. 2 illustrates crystal size distributions obtained by visual particle size analysis of paracetamol crystals formed from three separate CXF runs;

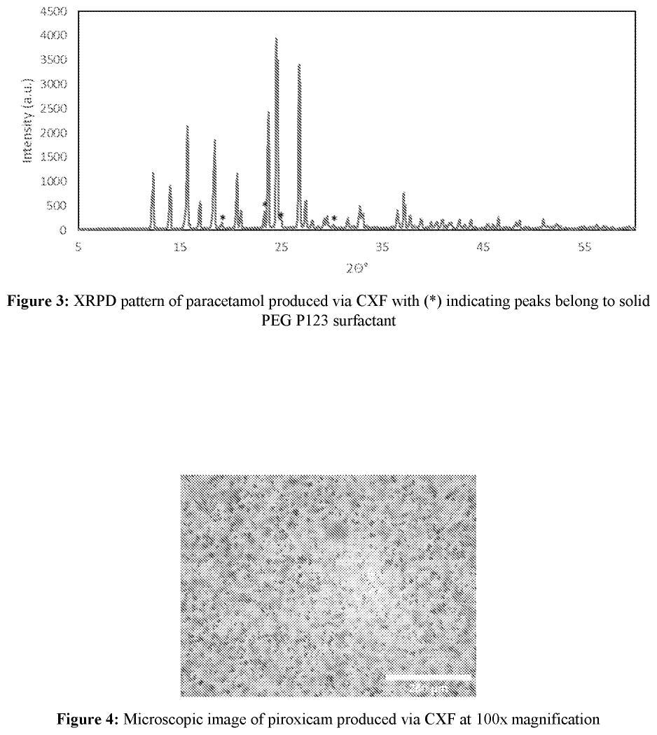

FIG. 3 illustrates the XRPD pattern of paracetamol produced via CXF with (*) indicating peaks belong to solid PEG P123 surfactant;

FIG. 4 illustrates a microscopic image of piroxicam produced via CXF at 100× magnification;

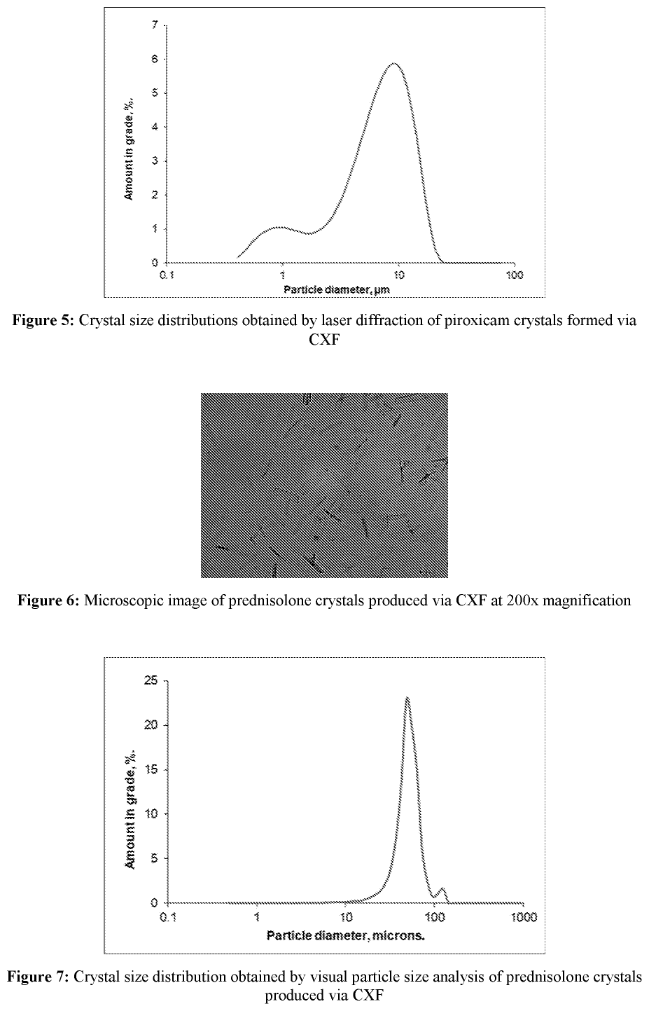

FIG. 5 illustrates crystal size distributions obtained by laser diffraction of piroxicam crystals formed via CXF;

FIG. 6 illustrates a microscopic image of prednisolone crystals produced via CXF at 200× magnification;

FIG. 7 illustrates crystal size distribution obtained by visual particle size analysis of prednisolone crystals produced via CXF;

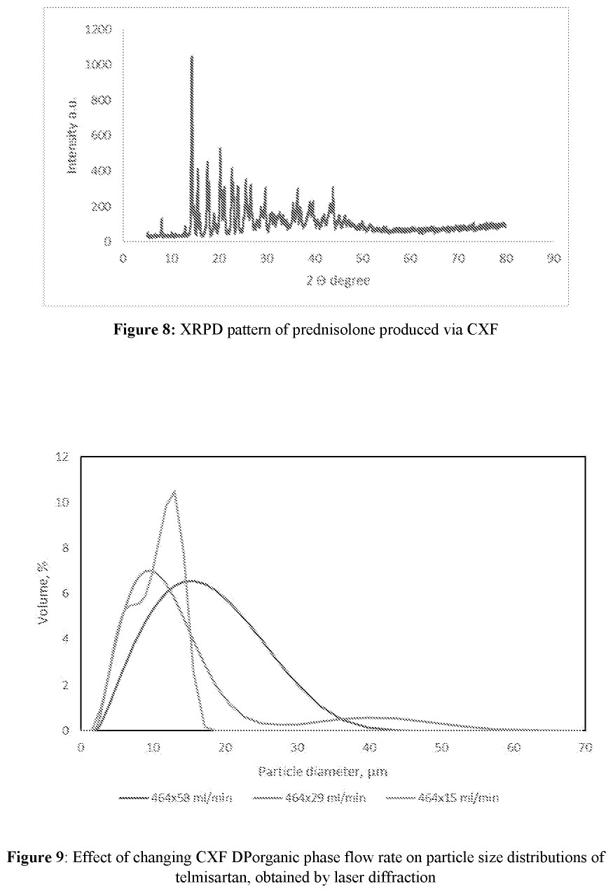

FIG. 8 illustrates an XRPD pattern of prednisolone produced via CXF;

FIG. 9 illustrates the effect of changing CXF DPorganic phase flow rate on particle size distributions of telmisartan, obtained by laser diffraction;

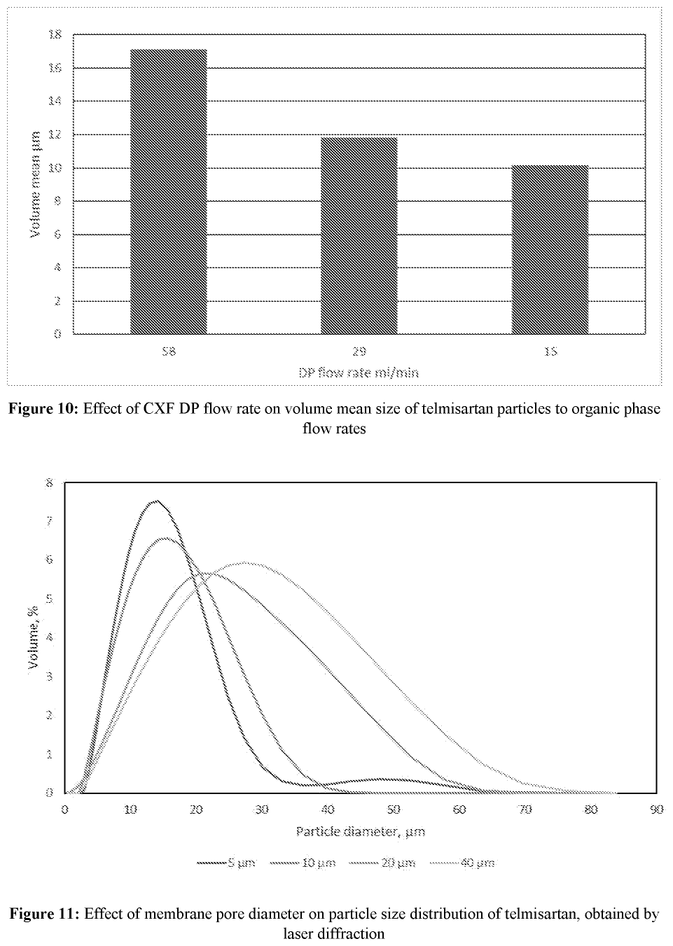

FIG. 10 illustrates the effect of CXF DP flow rate on volume mean size of telmisartan particles to organic phase flow rates;

FIG. 11 illustrates the effect of membrane pore diameter on particle size distribution of telmisartan, obtained by laser diffraction;

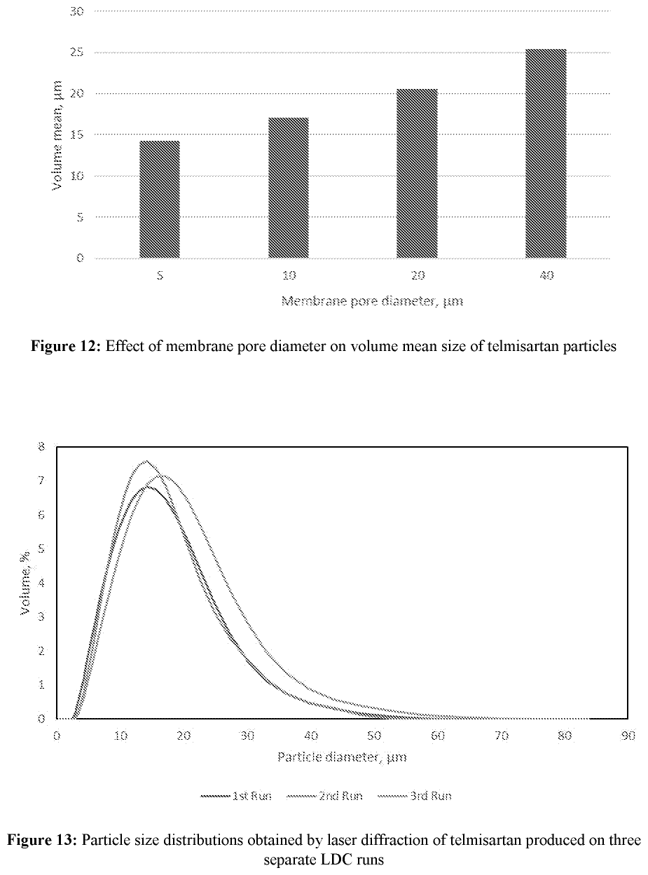

FIG. 12 illustrates the effect of membrane pore diameter on volume mean size of telmisartan particles;

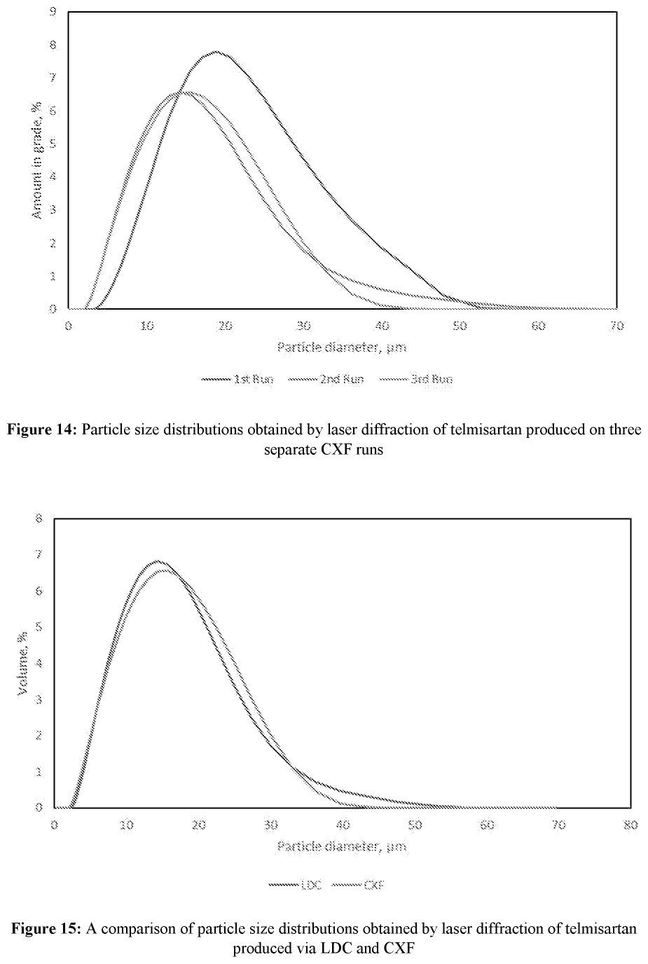

FIG. 13 illustrates particle size distributions obtained by laser diffraction of telmisartan produced on three separate LDC runs;

FIG. 14 illustrates particle size distributions obtained by laser diffraction of telmisartan produced on three separate CXF runs;

FIG. 15 illustrates a comparison of particle size distributions obtained by laser diffraction of telmisartan produced via LDC and CXF;

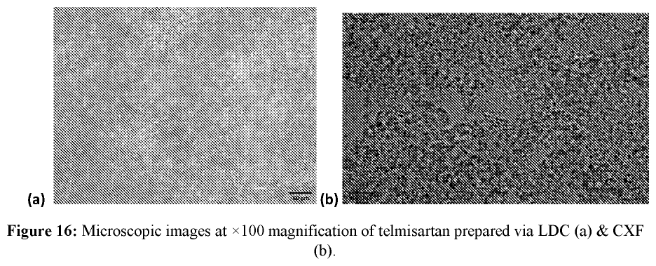

FIG. 16 illustrates microscopic images at ×100 magnification of telmisartan prepared via LDC (a) and CXF (b);

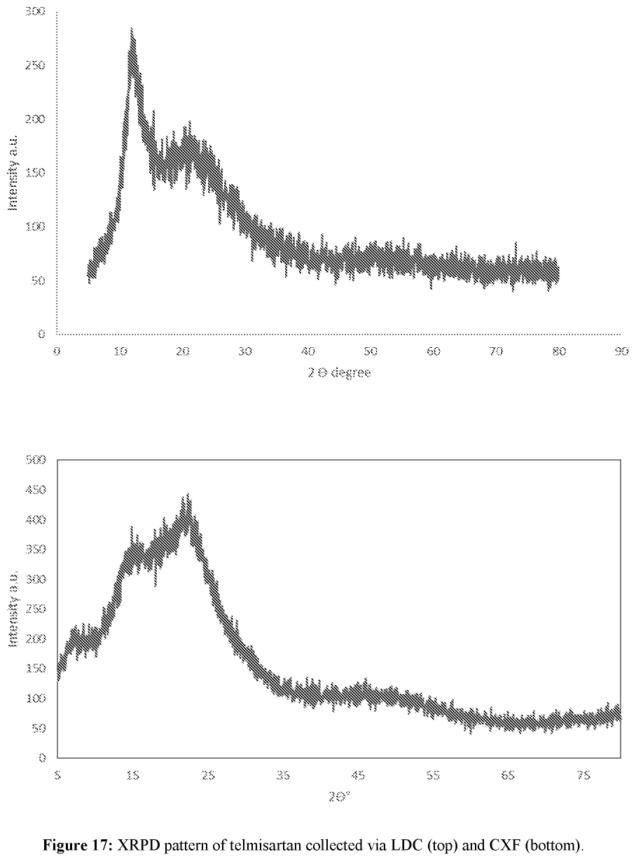

FIG. 17 illustrates an XRPD pattern of telmisartan collected via LDC (top) and CXF (bottom);

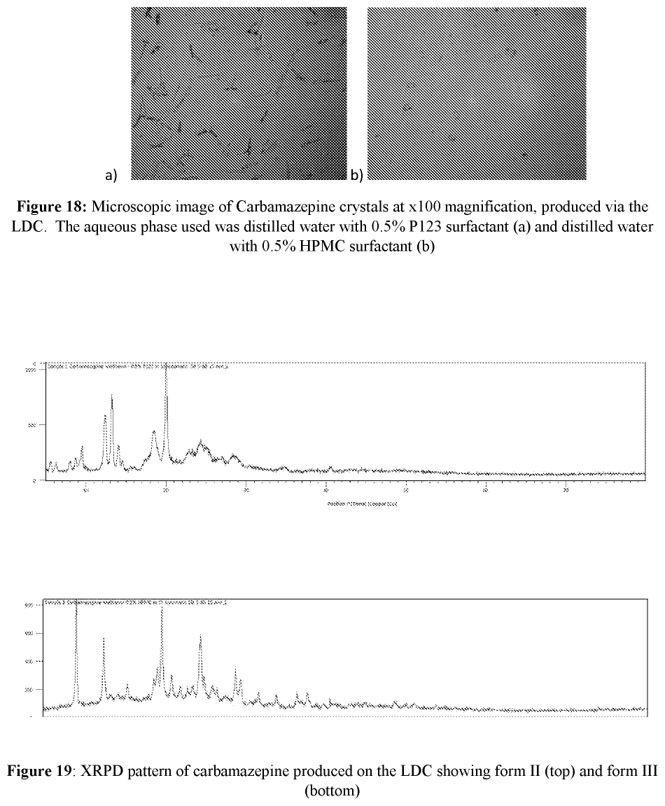

FIG. 18 illustrates a microscopic image of Carbamazepine crystals at ×100 magnification, produced via the LDC. The aqueous phase used was distilled water with 0.5% P123 surfactant (a) and distilled water with 0.5% HPMC surfactant (b);

FIG. 19 illustrates an XRPD pattern of carbamazepine produced on the LDC showing form II (top) and form III (bottom);

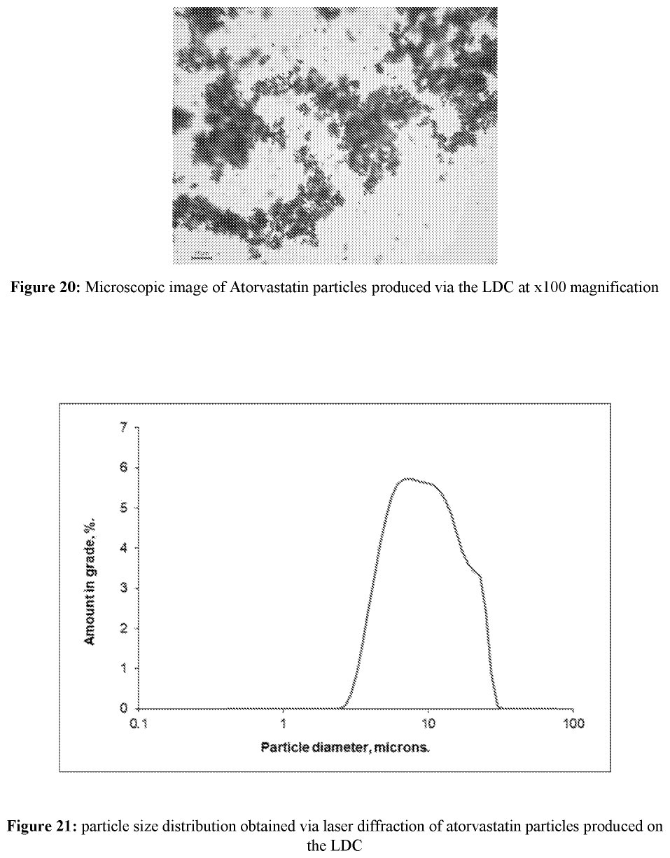

FIG. 20 illustrates a microscopic image of Atorvastatin particles produced via the LDC at ×100 magnification;

FIG. 21 illustrates particle size distribution obtained via laser diffraction of atorvastatin particles produced on the LDC;

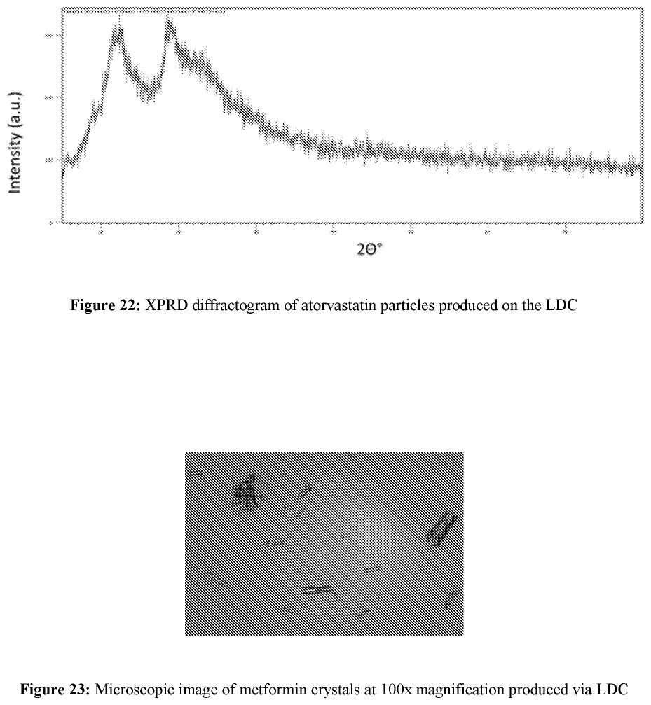

FIG. 22 illustrates an XPRD diffractogram of atorvastatin particles produced on the LDC; and

FIG. 23 illustrates a microscopic image of metformin crystals at 100× magnification produced via LDC.

### EXAMPLES

### Example 1

Reproducibility of the Production of Paracetamol Crystals Via CXF

A solution of paracetamol in ethanol (0.3 g/ml) was prepared, alongside an aqueous phase consisting of 0.5% wt. PEG P123 in DI water. The CXF was configured with a membrane with 10 μm pores, spaced 200 μm apart in a square grid. A 9.5 mm insert was used. The materials were pumped using gear pumps.

The aqueous continuous phase was pumped through the annulus at the center of the membrane. The organic disperse phase was pumped through the top port of the device calibrated at a rate of 75 m/min, through the membrane and into the flow of continuous phase at 200 ml/min.

The solution was collected in a beaker and stirred with an overhead stirrer at 500 rpm for 10-15 minutes.

The experiment was repeated 3 times. The resultant crystal suspension was examined via microscopy (see FIG. 1), and Jorin Visual Particle Size Analyser (ViPA) for crystal size and uniformity, (FIG. 2 and Table 1).

The filtered and dried paracetamol crystals were then collected and analysed using X-ray powder diffraction (XRPD), which indicated a highly crystalline material (FIG. 3).

TABLE 1

| | | | | Average ± |
|---|---|---|---|---|
| Volume Distribution | Run 1 | Run 2 | Run 3 | Standard Deviation |
| D10 (μm) | 24.12 | 25.18 | 22.13 | 23.81 ± 0.894 |
| D50 (μm) | 41.15 | 40.82 | 39.29 | 40.42 ± 0.573 |
| D90 (μm) | 74.81 | 67.77 | 62.36 | 68.31 ± 3.604 |
| Volume Mean | 45.37 | 44.28 | 41.17 | 43.61 ± 1.258 |
| D[4,3] μm | | | | |
| StDev (μm) | 19.09 | 16.10 | 15.96 | 17.05 ± 1.021 |
| Span | 1.232 | 1.044 | 1.024 | 1.100 ± 0.066 |
| CV % | 42.08 | 36.35 | 38.78 | 39.07 ± 1.660 |

### Example 2

Production of Piroxicam Crystals Via CXF

2.5 g of Piroxicam (PRX) was dissolved in 100 ml of ethanol in a beaker at 32° C. to make up the disperse phase, at a concentration of 25 mg/ml. 0.5% weight hydroxypropyl methylcellulose (HPMC) in deionised water was used as the continuous phase.

The CXF was configured with a membrane with 5 μm pores, spaced 45 μm apart in a square grid. A 9.5 mm insert was used. The materials were pumped using gear pumps.

The aqueous continuous phase was pumped at 400 m/min through the annulus at the center of the membrane. The organic disperse phase was pumped at 17 mL/min through the top port of the device, through the membrane and into the flow of continuous phase.

The solution was collected in a 250 ml beaker and stirred with an overhead stirrer at 500 rpm for 10-15 minutes. The crystals were analyzed via microscopy and Laser Diffraction (Beckmann Coulter LS-230). The results are relayed in FIGS. 4 and 5 and Table 2.

TABLE 2

| Volume Distribution | Value |
|---|---|
| D10 (μm) | 1.240 |
| D50 (μm) | 6.562 |
| D90 (μm) | 12.96 |
| Mean (μm) | 6.978 |
| SD (μm) | 4.307 |
| Span | 1.78 |
| CV (%) | 61.7 |

### Example 3

Preparation of Prednisolone Crystals Via CXF

8 g of Prednisolone was dissolved in 200 ml of ethanol in a beaker at 55° C. to make up the organic phase. 0.5% PVA (Mowiol 23-88) in DI water was used as the aqueous phase.

The CXF was configured with a membrane with 10 μm pores, spaced 200 μm apart in a square grid. A 9.5 mm insert was used. The materials were pumped using gear pumps, calibrated so the aqueous continuous phase flow was 230 m/min and the organic disperse phase flow was 28 mL/min.

The aqueous continuous phase was pumped through the annulus at the center of the membrane. The organic disperse phase, held at 55° C., was pumped through the top port of the device, through the membrane pores and into the flow of continuous phase.

The resultant solution was collected in a 500 ml beaker and stirred with an overhead stirrer at 500 rpm for 10-15 minutes. The crystals were analyzed via microscopy and Jorin ViPA. The results are relayed in FIGS. 6 and 7 and Table 3.

The filtered and dried crystals were analyzed by XRPD, (FIG. 8) and indicated a highly crystalline material, in the polymorph form II.

TABLE 3

| Volume Distribution | Value |
|---|---|
| D10 (μm) | 29.47 |
| D50 (μm) | 43.76 |
| D90 (μm) | 60.78 |
| Volume Mean (μm) | 45.73 |
| SD (μm) | 15.29 |
| Span | 0.72 |
| CV (%) | 33.44 |

### Example 4

Effect of Disperse Phase Flow Rate on the Size Distribution of Telmisartan Particles

A solution of telmisartan in DMSO (0.06 g/ml) was prepared as the organic phase. The aqueous phase was composed of distilled water. The CXF was configured with a membrane with 10 μm pores, spaced 200 μm apart in a square grid. A 9.5 mm insert was used. The materials were pumped using gear pumps.

The aqueous continuous phase was pumped through the annulus at the center of the membrane at 464 m/min. The organic disperse phase was pumped through the top port of the device at rates of 58, 29 and 15 m/min, through the membrane and into the flow of continuous phase that was calibrated.

The solution was collected in a beaker and stirred with an overhead stirrer at 500 rpm for 5-10 minutes. The crystals were analyzed via laser diffraction, and the results are reported in Table 4 and FIGS. 9 and 10.

TABLE 4

| Volume Distribution | 58 mL/min* | 29 mL/min | 15 mL/min |
|---|---|---|---|
| D10 (μm) | 7.359 | 4.981 | 4.653 |
| D50 (μm) | 15.66 | 10.09 | 10.11 |
| D90 (μm) | 28.00 | 18.9 | 15.76 |
| Volume Mean (μm) | 17.11 | 11.79 | 10.17 |
| S.D. (μm) | 8.565 | 7.943 | 4.179 |
| Span | 1.318 | 1.379 | 1.099 |

*Average of three

### Example 5

Effect of Membrane Pore Diameter on the Size Distribution of Telmisartan Particles

A solution of telmisartan in DMSO (0.06 g/ml) was prepared as the organic phase. The aqueous phase was composed of distilled water. The CXF was configured with membranes of different pore sizes that included 5, 10, 20 and 40 μm pores, spaced 200 μm apart in a square grid. A 9.5 mm insert was used. The materials were pumped using gear pumps.

The aqueous continuous phase was pumped through the annulus at the center of the membrane at a rate of 464 ml/min. The organic disperse phase was pumped through the top port of the device at a rate of 58 mL/min, through the membrane pores and into the flow of continuous phase.

The solution was collected in a beaker and stirred with an overhead stirrer at 500 rpm for 5-10 minutes. The crystals were analyzed via laser diffraction, and the results are reported in Table 5 and FIGS. 11 and 12.

TABLE 5

| Volume | 5 μm | 10 μm | 20 μm | 40 μm |
|---|---|---|---|---|
| Distribution | pore | pore* | pore | pore |
| D10 μm | 6.506 | 7.359 | 6.555 | 8.451 |
| D50 μm | 13.01 | 15.66 | 18.72 | 23.16 |
| D90 μm | 22.83 | 28.00 | 37.68 | 45.99 |
| Volume Mean μm | 14.31 | 17.11 | 20.60 | 25.42 |
| S.D. | 7.629 | 8.565 | 11.90 | 14.34 |
| Span μm | 1.255 | 1.318 | 1.663 | 1.621 |
| C.V % | 53.31 | 50.06 | 57.77 | 56.41 |

*Average of three

### Example 6

Reproducibility of the Production of Amorphous Telmisartan and Translation from Stirred Cell LDC to Continuous Crossflow CXF Devices A solution of telmisartan in DMSO (0.06 g/ml) was prepared for use as the organic phase. An aqueous phase of 50 ml of DI water was prepared. The LDC was configured with a membrane with 10 μm pores, spaced 200 μm apart in a square grid, in a ring sat underneath a stirrer paddle. A volume of 6 ml of DP was added at an injection rate of 10 ml/min. The CP and DP volume ratio is 8:1. The stirrer was set to 14V (1750 rpm). Once addition of the DP was complete the solution was stirred for 5-10 minutes.

The LDC runs were repeated three times.

The particle size distribution curves of the LDC runs were measured via laser diffraction and are shown in FIG. 13.

Additionally, a solution of telmisartan in DMSO (0.06 g/ml) was prepared as an organic phase. An aqueous phase was composed of distilled water. A CXF was configured with a membrane with 10 μm pores, spaced 200 μm apart in a square grid. A 9.5 mm insert was used. The materials were pumped using gear pumps.

The aqueous continuous phase was pumped through the annulus at the center of the membrane at a rate of 464 ml/min. The organic disperse phase was pumped through the top port of the device at a rate of 58 mL/min, through the membrane pores and into the flow of continuous phase. This preserved the 8:1 CP:DP ratio used in the LDC experiments and generated a similar shear profile.

The solution was collected in a beaker and stirred with an overhead stirrer at 500 rpm for 5-10 minutes. The CXF runs were repeated three times.

The particle size distribution curves of the CXF runs were measured via laser diffraction and are shown in FIG. 14.

The particle size distributions of material produced on the LDC are shown in Table 6, with errors given as the standard deviations, alongside those for the CXF runs. FIG. 15 shows a crystal size distribution curve from an LDC run alongside a CXF run showing the repeatability in size and distribution.

Microscope analysis of telmisartan particles produced on the LDC and the CXF was carried out, and an example is shown in FIG. 16.

Filtered and dried crystals produced by both methods were analyzed by XRPD, (FIG. 17) and indicated an amorphous particle morphology.

TABLE 6

| Volume Distribution | LDC | CXF |
|---|---|---|
| D10 (μm) | 6.952 ± 0.357 | 7.359 ± 1.096 |
| D50 (μm) | 14.32 ± 0.693 | 15.66 ± 1.558 |
| D90 (μm) | 26.43 ± 1.268 | 28.0 ± 2.091 |
| Volume Mean | 15.79 ± 0.755 | 17.11 ± 1.538 |
| D[4,3] (μm) | | |
| StDev (μm) | 8.066 ± 0.430 | 8.565 ± 0.275 |
| Span | 1.360 ± 0.028 | 1.389 ± 0.070 |
| CV (%) | 51.07 ± 0.937 | 50.57 ± 3.09 |

### Example 7

Effect of Aqueous Phase Surfactant on Crystal Morphology of Carbamazepine Produced Via LDC

0.4 g of Carbamazepine was dissolved in 10 ml methanol in a beaker at 45° C. to make up the organic phase. In one instance, 0.5% Pluronic PEG P123 (Mn˜5,800) in DI water was used as the aqueous phase. In another instance, 0.5% HPMC (Mn˜10,000) in DI water was used as the aqueous phase.

The LDC was configured with a membrane with 10 μm pores, spaced 200 μm apart in a ring formation. The stirrer speed was set at 14V (1750 rpm), and the organic phase injection rate set at 10 ml/min. The line was primed with organic phase and the aqueous was poured into the stirred cell. The organic phase addition was started and ended when 3 ml of DP was injected.

The solution was left to stir until precipitation was visible. The crystals were analyzed via microscopy and Jorin ViPA. The results are relayed in FIG. 18 and Table 7.

The filtered and dried crystals were analyzed by XRPD (FIG. 19), indicating that carbamazepine produced via LDC using an aqueous phase of distilled water with 0.5% P123 surfactant had produced crystalline material of form II, and using an aqueous phase of distilled water with 0.5% HPMC surfactant had produced crystalline material of form III.

TABLE 7

| Volume Distribution | 0.5% P123 | 0.5% HPMC |
|---|---|---|
| D10 (μm) | 29.92 | 28.90 |
| D50 (μm) | 42.37 | 44.92 |
| D90 (μm) | 64.09 | 78.16 |
| Volume mean (μm) | 45.79 | 48.84 |
| StDev | 16.13 | 18.64 |
| Span | 0.81 | 1.10 |
| CV (%) | 35.22 | 38.18 |

### Example 8

Preparation of Atorvastatin Particles Via LDC

0.6 g of Atorvastatin was dissolved in 10 ml methanol in a beaker at room temperature to make up the organic phase. 0.5% HPMC (Mn˜10,000) in DI water was used as the aqueous phase.

The LDC was configured with a membrane with 10 μm pores, spaced 200 μm apart in a square grid, in a ring. The stirrer speed was set at 14V (1750 rpm), and the organic phase injection rate set at 10 ml/min. The line was primed with organic phase and the CP was poured into the stirred cell. Addition was started and ended when 3 ml of DP was injected.

The solution was left to stir until precipitation was visible. The crystals were analyzed via microscopy and Laser Diffraction (Beckmann Coulter LS-230). The results are relayed in FIGS. 20 and 21, and Table 8.

The resulting solution was filtered and dried to obtain dry atorvastatin. This was analysed via XRPD and indicated an amorphous particle morphology (FIG. 22).

TABLE 8

| Volume Distribution | Value |
|---|---|
| D10 (μm) | 4.38 |
| D50 (μm) | 8.78 |
| D90 (μm) | 18.92 |
| Volume mean (μm) | 10.24 |
| StDev | 5.53 |
| Span | 1.66 |
| CV (%) | 54.0 |

### Example 9

Preparation of Metformin Crystals Via LDC

0.4 g of Metformin was dissolved in 10 ml methanol in a beaker to make up the organic phase. 0.5% Tween 20 in acetonitrile was used as the aqueous phase.

The LDC was configured with a membrane with 10 μm pores, spaced 200 μm apart in a ring-shape. The stirrer speed was set at 14V (1750 rpm), and the injection rate set at 10 ml/min. The line was primed with organic phase and the aqueous phase was poured into the dispersion cell. Addition of the organic phase was started and ended when 3 ml of DP was injected.

The solution was left to stir until precipitation was visible. The crystals were analyzed via microscopy and Jorin ViPA. The results are relayed in FIG. 23 and Table 9.

TABLE 9

| Volume Distribution | Value |
|---|---|
| D10 (μm) | 25.63 |
| D50 (μm) | 50.80 |
| D90 (μm) | 81.03 |
| Size Mean (μm) | 12.430 |
| Volume Mean (μm) | 53.10 |
| StDev | 22.04 |
| Span | 1.09 |
| CV (%) | 41.51 |

1. A method of preparing solid particles of a compound, said method comprising controlling provision of a liquid phase, wherein said liquid phase comprises a solution of the compound, in a first flow direction to a membrane, said membrane defining a plurality of pores; and controlling the supersaturation of the liquid phase after it has passed through the membrane via the plurality of pores, to form solid particles of the compound.

2. The method according to claim 1 wherein the supersaturation of the liquid is controlled by cooling the liquid phase, antisolvent precipitation or reverse antisolvent precipitation.

3. The method according to claim 2 wherein the supersaturation of the liquid is controlled by cooling the liquid phase after it has passed through the membrane.

4. The method according to claim 2 wherein the supersaturation of the liquid is controlled by antisolvent precipitation after the liquid phase has passed through the membrane.

5. The method according to claim 2 wherein the supersaturation of the liquid is controlled by reverse antisolvent precipitation after the liquid phase has passed through the membrane.

6. The method according claim 1 wherein the method comprises the preparation of solid particles of a compound in crystalline form or amorphous form, or a combination thereof.

7. (canceled)

8. (canceled)

9. A method of preparing solid particles of a compound, said method comprising dispersing a first liquid phase in a second liquid phase;
wherein said first liquid phase comprises a solution of the compound and said second liquid phase comprises an antisolvent; or said first liquid phase comprises an antisolvent and said second liquid phase comprises a solution of the compound;
said method comprising controlling provision of the first liquid phase in a first flow direction to a membrane, said membrane defining a plurality of pores; and controlling provision of the second liquid phase to the membrane in a crossflow to the first flow direction, via the plurality of pores, to form solid particles of the compound.

10. (canceled)

11. (canceled)

12. The method according to claim 9 wherein the method comprises the preparation of solid particles of a compound in crystalline form or amorphous form, or a combination thereof.

13. (canceled)

14. (canceled)

15. A method of preparing solid particles of a compound, said method comprising dispersing a first liquid phase in a second liquid phase;
wherein said first liquid phase comprises a solution of the compound and said second liquid phase comprises an antisolvent phase; or said first liquid phase comprises an antisolvent and said second liquid phase comprises a solution of the compound;
wherein said method uses a crossflow emulsification apparatus; said crossflow emulsification apparatus (AXF) comprising:
an outer tubular sleeve provided with a first inlet at a first end; a particle outlet; and a second inlet, distal from and inclined relative to the first inlet;
a tubular membrane provided with a plurality of pores and adapted to be positioned inside the tubular sleeve; and
optionally an insert adapted to be located inside the tubular membrane, said insert comprising an inlet end and an outlet end, each of the inlet end and an outlet end being provided with chamfered region; the chamfered region is provided with a plurality of orifices and a furcation plate; and
controlling provision of the first liquid phase to the tubular membrane; and controlling provision of a second liquid phase to the tubular membrane via the plurality of pores to form solid particles of the compound.

16. (canceled)

17. (canceled)

18. The method according to claim 15 wherein the method comprises the preparation of solid particles of a compound in crystalline form or amorphous form, or a combination thereof.

19. (canceled)

20. The method according to claim 18 wherein the method comprises the preparation of solid amorphous particles of a compound.

21. (canceled)

22. The method according to claim 15 wherein the solution comprises one or more dissolved organic compounds, the one or more dissolved organic compounds comprising pharmaceutically active compounds or drugs, bioactive agents, nutraceuticals, polymers and the like.

23. (canceled)

24. The method according to claim 1 wherein the compound is of low bioavailability.

25.-27. (canceled)

28. The method according to claim 22 wherein the solution comprises a pharmaceutically active compound is selected from one or more of antifungal agents, such as, itraconazole fluoconazole, terconazole, ketoconazole and saperconazole; anti-infective agents, such as griseofulvin and griseoverdin; antibiotics, such as, amoxicillin, azithromycin, cephalexin, cefixime, cefoperazone, ceftriaxone, ciprofloxacin, clarithromycin, clavulanic acid, clindamycin, doxycycline, erythromycin, gentamycin, levofloxacin, meropenem, metronidazole, neomycin, norfloxacin, ofloxacin, ornidazole, oxytctracycline, piperacillin, rifampicin, streptomycin, sulbactam, sulfadiazine, tazobactam, tetracycline and tinidazole; anti malaria drugs, such as, atovaquone and artesunate; protein kinase inhibitors, such as, afatinib, axitinib, bosutinib, cetuximab.crizotinib, dasatinib, erlotinib, fostamatinib, gefitinib, ibrutinib, imatinib, zemurasenib, lapatinib, lenvatinib, mubritinib and nilotinib; immune system modulators, such as, cyclosporine; cardiovascular drugs, such as, digoxin and spironolactone; sterols or steroids, such as, betamethasone; ACE inhibitors, such as, captopril, enalapril, ramipril, quinapril, perindopril, lisinopril, and fosinopril; adenohypophyseal hormones; adrenergic antagonists, such as, phentolamine, phenoxybenzamine, tamsulosin, propranolol, atenolol, metoprolol, timolol and acebutolol; adrenocortical steroids; inhibitors of the biosynthesis of adrenocortical steroids; alpha-adrenergic agonists, such as methoxamine, phenylephrine, methyldopa, norepinephrine; alpha-adrenergic antagonists, such as, phentolamine and phenoxybenzamine; analgesics, such as, aspirin and paracetamol; antipyretics and anti-inflammatory agents, such as, diclofenac, ibuprofen, naproxen and ketoprofen; androgens, local anaesthetics, such as, lidocaine; antiaddictive agents; antiandrogens; antiarrhythmic agents, such as, verapamil and diltiazem; antiasthmatic agents, such as, beclomethasone, budesonide, fluticasone, reproterol, salbutamol and salmeterol; anticholinergic agents, such as, ipratropium and oxybutynin; anticholinesterase agents, such as, donepezil; anticoagulants, such as, dabigatran, rivaroxaban, apixaban, edoxaban and betrixaban; antidiabetic agents, such as, metformin; antidiarrheal agents; antidiuretics; antiemetic and prokinetic agents; antiepileptic agents, such as carbamazepine, gabapentin oxcarbazepine; antiestrogens; antifungal agents; antihypertensive agents, such as, losartan, olmesartan, telmisartan and valsartan; antimicrobial agents; antimigraine agents, such as, zolmitriptan; antimuscarinic agents; antineoplastic agents; antiparasitic agents; antiparkinsons agents, such as, carbidopa and levodopa; antiplatelet agents; antiprogestins; antithyroid agents; antitussives; antiviral agents; antidepressants; azaspirodecanediones; barbiturates; benzodiazepines; benzothiadiazides; beta-adrenergic agonists; beta-adrenergic antagonists; selective adrenergic antagonists; selective agonists; bile salts; butyrophenones; calcium channel blockers; catecholamines and sympathomimetic drugs; cholinergic agonists; cholinesterase reactivators; cognitive enhancers, such as, piracetam; dermatological agents; diphenylbutylpiperidines; diuretics; ergot alkaloids; oestrogens; ganglionic blocking agents; ganglionic stimulating agents; glucocorticoid steroids, such as, dexamethasone and prednisolone; agents for control of gastric acidity and treatment of peptic ulcers; haematopoietic agents; histamines; antihistamine; HMG-CoA reductase inhibitors, e.g. statins, such as, atorvastatin, fluvastatin, lovastatin, pitavastatin, pravastatin, rosuvastatin and simvastatin; 5-hydroxytryptamine antagonists; hypnotics and sedatives; immunosuppressive agents; laxatives; methylxanthines; monoamine oxidase inhibitors; neuromuscular blocking agents; nutrients or dietary supplements, such as, vitamin B1, vitamin B6 and retinol; organic nitrates; opioid analgesics and antagonists; pancreatic enzymes; phenothiazines; progestins; prostaglandins; agents for the treatment of psychiatric disorders; retinoids; sodium channel blockers; thrombolytic agents; thyroid agents; tricyclic antidepressants; tyrosine kinase inhibitors, such as, axitinib, crizotinib, dasatinib, erlotinib, gefitinib, imatinib, lapatinib, nilotinib, pazopanib, regorafenib, ruxolitinib, sorafenib, sunitinib, vandetanib and vemurafenib; drugs from the group comprising danazol, acyclovir, dapsone, indinavir, lopinavir, nifedipine, nitrofurantion, phentytoin, ritonavir, saquinavir, sulfamethoxazole, valproic acid, trimethoprin, acetazolamide, azathioprine, iopanoic acid, nalidixic acid, nevirapine, praziquantel, rifampicin, albendazole, amitrptyline, artemether, lumefantrine, chloropromazine, clofazimine, efavirenz, iopinavir, folic acid, glibenclamide, haloperidol, ivermectin, mebendazole, niclosamide, pyrantel, pyrimethamine, sulfadiazine, sulfasalazine, triclabendazole, and cinnarizine; and combinations thereof.

29. (canceled)

30. (canceled)

31. The method according to claim 28 wherein the pharmaceutically active compound is an antihypertensive agent, such as, telmisartan.

32. The method according to claim 28 wherein the pharmaceutically active compound is an antihypertensive agent in amorphous form, such as, telmisartan.

33.-39. (canceled)

40. The method according to claim 15 wherein the apparatus includes an insert.

41.-77. (canceled)

78. The method according to claim 1 wherein the method is a continuous process.

79. (canceled)

80. (canceled)

81. A solid particle prepared by the method according to claim 1.

82. A solid particle according to claim 81 wherein the particle comprises an active agent.

83. A solid particle according to claim 81 wherein the solid particles are in crystalline form or amorphous form, or a combination thereof.

84.-89. (canceled)

90. A solid particle according to claim 81 wherein the active agent is selected from one or more of antifungal agents, such as, itraconazole fluoconazole, terconazole, ketoconazole and saperconazole; anti-infective agents, such as griseofulvin and griseoverdin; antibiotics, such as, amoxicillin, azithromycin, cephalexin, cefixime, cefoperazone, ceftriaxone, ciprofloxacin, clarithromycin, clavulanic acid, clindamycin, doxycycline, erythromycin, gentamycin, levofloxacin, meropenem, metronidazole, neomycin, norfloxacin, ofloxacin, ornidazole, oxytctracycline, piperacillin, rifampicin, streptomycin, sulbactam, sulfadiazine, tazobactam, tetracycline and tinidazole; anti malaria drugs, such as, atovaquone and artesunate; protein kinase inhibitors, such as, afatinib, axitinib, bosutinib, cetuximab.crizotinib, dasatinib, erlotinib, fostamatinib, gefitinib, ibrutinib, imatinib, zemurasenib, lapatinib, lenvatinib, mubritinib and nilotinib; immune system modulators, such as, cyclosporine; cardiovascular drugs, such as, digoxin and spironolactone; sterols or steroids, such as, betamethasone; ACE inhibitors, such as, captopril, enalapril, ramipril, quinapril, perindopril, lisinopril, and fosinopril; adenohypophyseal hormones; adrenergic antagonists, such as, phentolamine, phenoxybenzamine, tamsulosin, propranolol, atenolol, metoprolol, timolol and acebutolol; adrenocortical steroids; inhibitors of the biosynthesis of adrenocortical steroids; alpha-adrenergic agonists, such as methoxamine, phenylephrine, methyldopa, norepinephrine; alpha-adrenergic antagonists, such as, phentolamine and phenoxybenzamine; analgesics, such as, aspirin and paracetamol; antipyretics and anti-inflammatory agents, such as, diclofenac, ibuprofen, naproxen and ketoprofen; androgens, local anaesthetics, such as, lidocaine; antiaddictive agents; antiandrogens; antiarrhythmic agents, such as, verapamil and diltiazem; antiasthmatic agents, such as, beclomethasone, budesonide, fluticasone, reproterol, salbutamol and salmeterol; anticholinergic agents, such as, ipratropium and oxybutynin; anticholinesterase agents, such as, donepezil; anticoagulants, such as, dabigatran, rivaroxaban, apixaban, edoxaban and betrixaban; antidiabetic agents, such as, metformin; antidiarrheal agents; antidiuretics; antiemetic and prokinetic agents; antiepileptic agents, such as carbamazepine, gabapentin oxcarbazepine; antiestrogens; antifungal agents; antihypertensive agents, such as, losartan, olmesartan, telmisartan and valsartan; antimicrobial agents; antimigraine agents, such as, zolmitriptan; antimuscarinic agents; antineoplastic agents; antiparasitic agents; antiparkinsons agents, such as, carbidopa and levodopa; antiplatelet agents; antiprogestins; antithyroid agents; antitussives; antiviral agents; antidepressants; azaspirodecanediones; barbiturates; benzodiazepines; benzothiadiazides; beta-adrenergic agonists; beta-adrenergic antagonists; selective adrenergic antagonists; selective agonists; bile salts; butyrophenones; calcium channel blockers; catecholamines and sympathomimetic drugs; cholinergic agonists; cholinesterase reactivators; cognitive enhancers, such as, piracetam; dermatological agents; diphenylbutylpiperidines; diuretics; ergot alkaloids; oestrogens; ganglionic blocking agents; ganglionic stimulating agents; glucocorticoid steroids, such as, dexamethasone and prednisolone; agents for control of gastric acidity and treatment of peptic ulcers; haematopoietic agents; histamines; antihistamine; HMG-CoA reductase inhibitors, e.g. statins, such as, atorvastatin, fluvastatin, lovastatin, pitavastatin, pravastatin, rosuvastatin and simvastatin; 5-hydroxytryptamine antagonists; hypnotics and sedatives; immunosuppressive agents; laxatives; methylxanthines; monoamine oxidase inhibitors; neuromuscular blocking agents; nutrients or dietary supplements, such as, vitamin B1, vitamin B6 and retinol; organic nitrates; opioid analgesics and antagonists; pancreatic enzymes; phenothiazines; progestins; prostaglandins; agents for the treatment of psychiatric disorders; retinoids; sodium channel blockers; thrombolytic agents; thyroid agents; tricyclic antidepressants; tyrosine kinase inhibitors, such as, axitinib, crizotinib, dasatinib, erlotinib, gefitinib, imatinib, lapatinib, nilotinib, pazopanib, regorafenib, ruxolitinib, sorafenib, sunitinib, vandetanib and vemurafenib; drugs from the group comprising danazol, acyclovir, dapsone, indinavir, lopinavir, nifedipine, nitrofurantion, phentytoin, ritonavir, saquinavir, sulfamethoxazole, valproic acid, trimethoprin, acetazolamide, azathioprine, iopanoic acid, nalidixic acid, nevirapine, praziquantel, rifampicin, albendazole, amitrptyline, artemether, lumefantrine, chloropromazine, clofazimine, efavirenz, iopinavir, folic acid, glibenclamide, haloperidol, ivermectin, mebendazole, niclosamide, pyrantel, pyrimethamine, sulfadiazine, sulfasalazine, triclabendazole, and cinnarizine; and combinations thereof.

91. (canceled)

92. (canceled)
